(19) United States
(12) Patent Application Publication
(10) Pub. No.: US 2024/0325304 A1
(43) Pub. Date: Oct. 3, 2024

(54) Medication for Alleviating Parkinson's Symptoms and Method of Administering

(71) Applicant: Greg Eckles, Portland, OR (US)

(72) Inventor: Greg Eckles, Portland, OR (US)

(21) Appl. No.: 18/733,769

(22) Filed: Jun. 4, 2024

Related U.S. Application Data
(62) Division of application No. 16/950,189, filed on Nov. 17, 2020, now abandoned.

(51) Int. Cl. A61K 9/08; A61K 9/00; A61K 31/10; A61K 33/00

(52) U.S. Cl. CPC A61K 9/08; A61K 9/0043; A61K 9/0078; A61K 31/10; A61K 33/00

(57) ABSTRACT

A drug and method of administering it to temporarily moderate the symptoms of neurological degenerative diseases such as Parkinson's. The drug is mixed in a liquid form with a nasal atomizer and administered twice a day by introduction as an atomized liquid into the nasal cavity. It provides instantaneous moderation of tremors, internal uneasiness, limb rigidity, gait and balance problems, memory loss, slurred speech and verbal trailing off. The results last approximately three to four hours. The dosage is dependent on the weight of the patient and is given upon rising in the morning and at bed time.

### FIELD

The present disclosure relates, in general, to short term alleviation of the symptoms of neurological degenerative diseases, primarily Parkinson's Disease, and more particularly to a new drug and novel method of administering it.

### BACKGROUND

Parkinson's disease is a long-term degenerative disorder of the central nervous system that primarily affects the motor system. The motor symptoms include tremors, limb rigidity, slowness of movement, difficulty with walking (gait and balance problems) and speech difficulty (stuttering, mumbles, slurs and trailing off). This is a result from the death of cells in the substantia nigra, a region of the midbrain, leading to a dopamine deficit. The cause of this cell death is poorly understood, but involves the build-up of proteins into Lewy bodies in the neurons. Parkinson's disease dementia is common in the advanced stages of the disease as well as depression and anxiety.

There is no cure for Parkinson's disease and current treatments aim only to improve the symptoms. To date there is no good long term solution to alleviate the symptoms. Levodopa (L-DOPA) is usually the first treatment medicine, however this tends to have a short term of effectiveness and is commonly followed by dopamine agonists. As the disease progresses, these medications become less effective, while at the same time producing a side effect marked by involuntary muscle movements and compulsive behaviors. In severe cases with extreme tremors and shaking, surgery to place microelectrodes for deep brain stimulation has been used to reduce these motor symptoms. The motor related symptoms are not necessarily continual and can present quickly and intensify for brief periods.

Henceforth, a medication and method of application that would help reduce Parkinson's symptoms quickly, without side effects would fulfill a long felt need in the neurological degenerative diseases industry. This new invention utilizes and combines known chemicals into a new medication with a novel method of application that has unexpected results in overcoming the symptoms of Parkinson's, and avoids the prior art's aforementioned problems with long term ineffectiveness and side effects.

### BRIEF SUMMARY

In accordance with the embodiment presented, an inexpensive, easily administered medication for the continued short term alleviation of the symptoms of Parkinson's disease is provided.

Various modifications and additions can be made to the embodiment discussed without departing from the scope of the invention. For example, while the embodiment described above refers to particular features, the scope of this invention also includes embodiments having different combination of features and embodiments that do not include all of the above described features.

### BRIEF DESCRIPTION OF THE DRAWINGS

A further understanding of the nature and advantages of particular embodiments may be realized by reference to the remaining portions of the specification and the drawings, in which like reference numerals are used to refer to similar components.

FIGS. 1 to 5 are representative drawings of the steps in the creation, bottling and administration of the medication;

FIGS. 1 and 2 are representative drawings of the creation of the medication by the combination of the drug's constituents;

FIG. 3 is a representative drawing of the medication being placed into its delivery apparatus;

FIG. 4 is a representative drawing of the prescribing doctor's prescription for the medication; and

FIG. 5 is a representative drawing of the administration of the medication.

### DETAILED DESCRIPTION OF CERTAIN EMBODIMENTS

While various aspects and features of have been summarized above, the following detailed description illustrates the preferred embodiment in further detail to enable one skilled in the art to make and use such. The described examples are provided for illustrative purposes and are not intended to limit the scope of the invention.

In the following detailed description, numerous specific details are set forth to enable a thorough understanding of the inventive concept. It should be understood, however, that persons having ordinary skill in the art may practice the inventive concept without these specific details. In other instances, well-known methods, procedures and components, have not been described in detail so as not to unnecessarily obscure aspects of the embodiments. It will be apparent to one skilled in the art, however, that other embodiments of the present invention may be practiced without some of these specific details.

Unless otherwise indicated, all numbers herein used to express quantities, volumes or concentrations should be understood as being modified in all instances by the term “approximately” meaning plus or minus 25%.

As used herein, the term “biofilms” refers to a collective of one or more types of microorganisms that can grow on many different surfaces, including the human brain. Microorganisms that form biofilms include bacteria, fungi and protists.

The present invention relates to a novel design for a medication to temporarily yet almost instantly, partially alleviate the symptoms of neurological degenerative diseases, primarily Parkinson's Disease. Specifically, it provides an unexpected result in that it provides instantaneous moderation of tremors, internal uneasiness, limb rigidity, gait and balance problems, memory loss, slurred speech and verbal trailing off. It is a fluid medication sprayed into the nasal cavity with an atomization means as an atomized fluid. Its effects last about three to four hours and are almost instantaneous.

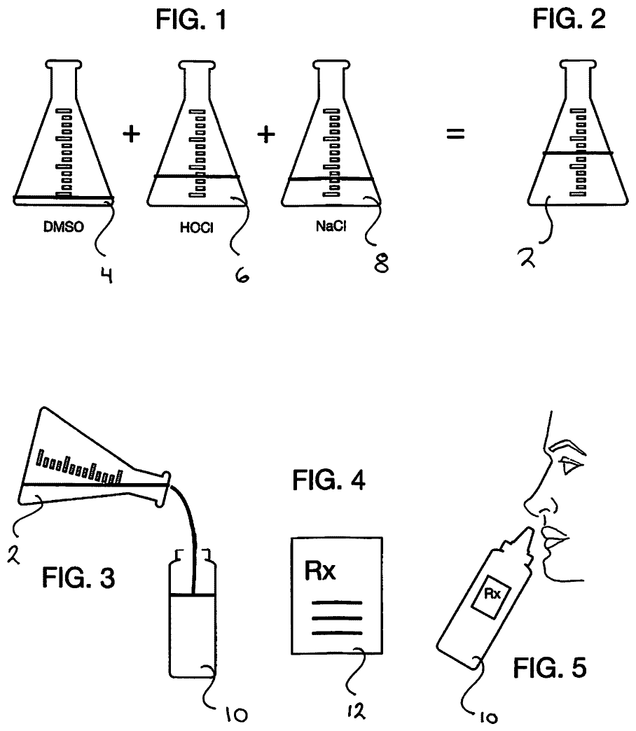

Looking at FIGS. 1 and 2, it can be seen that the medication 2 is prepared by introducing one ml of DMSO 4 and 30 ml of HOCl 6, (a 1:30 ratio) in the concentrations set forth below, then diluting the mixture with 28 ml of a normalized saline solution 8 to a total volume of two fluid oz. (Two fluid ounces is the standard volume for a nasal spray delivery means 10.) Homogenously mixing the three ingredients will yield the medication in a concentration that is dose appropriate and able to be atomized with a standard manual nasal medication atomizer 10. (Note, these will deliver approximately 120 atomized sprays from this 2 fluid ounce volume.)

The primary two active ingredients in the medication are Dimethyl Sulfoxide (DMSO) and HOCl. Dimethyl sulfoxide (DMSO) is a highly polar organic reagent that has exceptional solvent properties for organic and inorganic chemicals. It is routinely used in molecular biology, especially in the polymerase chain reaction (PCR), in transformation and transfection, for cell lysis, and in cytofluorimetric assessment. In the medication DMSO is used at a 70% concentration, diluted down with distilled water from its reagent grade concentration of pure (99.9%) DMSO.

DMSO has a molecular formulae of C₂H₆OS or (CH₃)₂SO. It is a highly polar organic liquid used widely as a chemical solvent and a free radical scavenger. It shows a range of pharmacological activity including analgesia and anti-inflammation. It has two functions in the medication. First, because of its ability to penetrate biological membranes, it is used as a chaperone molecule to carry the HOCl into the cellular structure, and second, it also reconfigures the proteins that have been misfolded (deactivates prions) and acts as an anti-inflammatory for the brain.

Hypochlorous acid has a molecular formulae of HOCl or HClO. It is a weak acid typically formed when chlorine dissolves in water. It is produced in the human body by the immune cells to fight infections, as it acts against a wide range of microorganisms as it readily reacts with a variety of organic molecules and biomolecules including DNA, RNA, fatty acid groups, cholesterol, well as all nucleotides in vitro, and proteins. It has antimicrobial properties. HOCl is known to cause post-translational modifications to proteins, the notable ones being cysteine and methionine oxidation. A recent examination of HOCl's bactericidal role revealed it to be a potent inducer of protein aggregation. It is a more powerful oxidant that kills bacteria, bacterial spores, and viruses 100 times more effectively than chlorine bleach. Studies have shown its effectiveness in removing Pseudomonas aeruginosa biofilms. The Hypochlorous Acid 6 is used at a 100% equilibrium concentration in formulating the medication 2 and is used as an electrostatically bound stabilized hypochlorous acid such that it is stabilized (in equilibrium) in its solution.

The normalized saline solution 8 as used in the preferred embodiment medication is made of electrolyzed water and a 0.7 percent by weight NaCl concentration. (Although this concentration could be substituted with one between 0.5-1.5% by weight NaCl.) Although described herein in its preferred embodiment diluted with the saline solution to almost 50% of its initial concentration, it is known that the active constituents of the medication are the DMSO and HOCl and they can be administered undiluted or diluted in various concentrations with the saline solution or plain distilled water.

Once mixed, the medication is placed into a nasal atomizer 10 as seen in FIG. 3. It is to be noted that even with the dilute concentration of DMSO 4 in the medication 2, it is recommended that a glass, airtight bottle be employed with a spray atomizer pump made of a plastics like PETE, HDPE, LDPE, PTFE or PP. Other plastics such as PVC, PS and poly carbonate are soluble in DMSO and if used could contaminate the medication introducing unwanted chemicals at a cellular level to the patient.

After patient diagnosis of Parkinson's Disease or another neurodegenerative disorder by a qualified doctor, a prescription 12 is written (FIG. 4) that provides for 2 nasal pump sprays up the patient's nose upon rising and at bedtime, unless the patient is less than 120 pounds in bodyweight, when only 1 spray two times a day is prescribed. A standard nasal spray pump container holds two ounces of fluid and disperses approximately 0.4 ml of atomized fluid per pump. (Approximately here denotes + or −0.2 ml.)

The method of introduction of the medication is as an atomized nasal spray (FIG. 5) which is adsorbed through the cell membrane of the nasal epithelium and enters the olfactory through the olfactory nerve tract which is cranial nerve one to the cribriform plate down the olfactory nerve to the olmigdila (deep brain structure) and the memory portion of the brain which ends up in the brain stem. Upon adsorption of the medication 2 the cribriform process clears room out and allows the brain to regenerate. Once adsorbed in the brain, the medication can eradicate biofilms, a collection of types of microorganisms such as bacteria, viruses, mold, fungi and protists that grow on different surfaces of the human brain. (Often these masks themselves so the human body cannot put forth an immune response.)

Neuro degeneration is not just lack of oxygen it is multi-factorial. It is neuro toxicity caused by pesticides, chemicals, toxicants and heavy metals that have all bioaccumulated into the brain causing and triggering proteins to misfold and connect to the central nervous system leading to neuronal cell death. The spray synergistically works on autophagying (clearing dead cells by self-consumption), brain detoxification, halting the transfiguration and misconfiguration of the prionic activity of proteins, clearing misfolded proteins from the brain and removing biofilm for brain surfaces. Once adsorbed into the brain, this medication clears room out and allows the brain to regenerate and accept more oxygen. The medication seems to work well without any diminishment of the symptomatic relief or need for dosage increase to date. It has also been shown to reduce systemic inflation, orthopedic pain and nerve pain in the body, slow tremors and improve speech in Parkinson's and certain Creutzfeldt-Jakob Disease patients.

Clinical testing is performed with the medication stored in a glass container atomization means, and formulated as indicated below in its preferred embodiment of two fluid ounce volumes by mixing:
1 ml of DMSO—70% concentration (99.9% pure C₂H₆OS diluted to 70% with distilled H₂O);
30 ml of HOCL—equilibrium concentration 0.02 to 0.035% Hypochlorous acid (made of >97.796% electrolyzed H₂O+1.7 to 2.2% NaCl) electrostatically bound hypochlorous acid
28 ml of Normalized Saline Solution-distilled electrolyzed H₂O+0.7 to 0.9% NaCl

Although data is still being collected assembled from clinical testing, the following four short case studies are presented.

Patient A—was a 55 year old man presenting with a Marcons MERSA infection in his nasal cavity. Previous different antibiotic treatments proved fruitless as it was an antibiotic resistant. The patient developed chronic sinusitis. He was given the two times a day nasal administration of the medication, and in six weeks there was complete eradication of the negative Stafficcoci.

Patient B—was a 68 year old man that had been presenting with Parkinson's Disease, Aphasia and Dysphasia for approximately 2 years. His speech was mumbled, trailed off in weakness and slurred. After each two spray dose of the medication there is almost instantaneous noticeable improvement in the clarity of the speech. However, the noticeable improvements only lasts for 3 to 4 hours after the introduction of the medication. Repeated administrations of the medication showed no outward signs of lessening of the medication's effectiveness.

Patient C—was a 71 year old woman with Parkinson's Disease. She had an inability to speak and her walking coordination was off, symptomized by stuttered steps, freezing of her leg muscles and basic incoordination. She had a propensity for uncontrollably falling forward on the balls of her feet. Almost immediately after introduction of the medicine, she was able to speak, and her rigid gate and stutter step became much more fluid. Again, the remedial effects lasted only between three and four hours.

Patient D—was a 64 year old man with Parkinson's Disease. He had tremors on one side of his body which would slowly progress to the entire body. These external tremors were preceded by an inner tremor. Writing with a pen was impossible. Almost immediately after the introduction of the medication by two nasal spray atomization, the tremors reduced to the point where he could again write. The patient reported a calming of the inner tremor as well, although the results lasted only three to four hours.

The medication is inexpensive, easy to prepare and immediately relieves the physical symptoms of neurodegenerative disorders (primarily Parkinson's Disease) for approximately three to four hours.

While certain features and aspects have been described with respect to the preferred embodiment, one skilled in the art will recognize that numerous modifications are possible. The key components in this medication are the combined DMSO and the HOCl which can be administered as an atomized pump mist into the nose, either undiluted or diluted to various concentrations with a normalized saline solution. Moreover, while the procedure of the process for making the medication is detailed herein, unless the context dictates otherwise, various procedures may be reordered, added, and/or omitted in accordance with various embodiments. Consequently, it will be appreciated that the invention is intended to cover all modifications and equivalents within the scope of the following claims. What is claimed as the invention, therefore, is all such modifications as may come within the scope and spirit of the following claims and equivalents thereto.

1. A method of for the temporary treatment of a patient with a neurological degenerative disease, comprising the steps of:
mixing a homogeneous solution of 1 part dimethylsulfoxide (DMSO) mixed to 70% concentration with distilled water, 30 parts electrolyzed anhydrous chloride (HOCl) of a 0.02 to 0.035% concentration, made of more than 97.796% electrolyzed H₂O and 1.7 to 2.2% NaCl, and 28 parts normalized saline solution having a concentration of 0.7-0.9 percent NaCl with distilled water;
putting said homogeneous solution into a nasal spray delivery means that is made of materials that are not soluble in DMSO;
administering at least one atomized dose of said homogeneous solution of approximately 0.4 ml into each nasal cavity of a patient.

2. The method of claim 1 wherein the number of atomized doses administered to said patient is two when said patient is over 120 lbs in weight, and one when the patient is less than 120 lbs in weight.

3. The Method of claim 1 wherein said homogeneous solution is administered twice a day, once upon rising and once prior to sleep.
